(19) United States
(12) Patent Application Publication
(10) Pub. No.: US 2008/0045859 A1
(43) Pub. Date: Feb. 21, 2008

(54) Devices and Methods for In-Vivo Pathology Diagnosis

(76) Inventors: Michael H. Fritsch, Lincoln, NE (US); John H. Fritsch, Lincoln, NE (US); Josephine M. Fritsch, Lincoln, NE (US)

Correspondence Address: INDIANO VAUGHAN LLP, SUITE 1300, ONE NORTH PENNSYLVANIA STREET, INDIANAPOLIS, IN, 46204, US

(21) Appl. No.: 11/840,438

(22) Filed: Aug. 17, 2007

Related U.S. Application Data
(60) Provisional application No. 60/838,614, filed on Aug. 19, 2006.

(51) Int. Cl. A61B 10/04; A61B 1/00

(52) U.S. Cl. 600/567; 600/121

(57) ABSTRACT

An in vivo pathology diagnosis system includes a penetrating device that is at least one of an endoscope sheath and an endoscope, the penetrating device having irrigation and working channels with openings at a distal end chamber of the device. The device is structured for penetrating tissue until the distal end is proximate a target tissue area. A method includes supplying stain to the chamber via the irrigation channels, thereby staining the target tissue area. Pathology of the stained target tissue is performed by viewing such stained tissue through the penetrating device. The viewing may be performed through a selected lens segment of a tissue contact type endoscope or endoscope sheath. A suction channel may be provided for removing material from the chamber, and cauterizing electrodes may be provided as part of the device. Control of in vivo staining may be performed by a computer program.

### RELATED APPLICATIONS

This application claims benefit of priority from Fritsch et al., U.S. Provisional Patent Application Ser. No. 60/838,614 filed on 19 Aug. 2006, which is incorporated herein by reference in its entirety.

### I. FIELD OF THE INVENTION

The invention relates generally to devices and methods of endoscopy and, more particularly, to devices and methods for accessing and diagnosing pathologic histologic tissue including deep tissue.

### II. BACKGROUND OF THE INVENTION

Physicians who practice pathology conventionally diagnose and characterize disease in living patients by examining biopsies and other specimens. Such examination of tissues and cells has generally involved gross and microscopic visual examination of tissues, with special stains and immunohistochemistry, and other procedures, being employed to visualize specific proteins and other substances in and around cells.

When certain surgeries are performed, a corresponding typical pathology histology diagnosis of a patient's tissue may be achieved according to one or more of a limited number of standard procedures. Two of these primary histologic diagnostic procedures are “permanent-section” and “frozen-section,” and such may include, for example, a fixation process for preserving cell structure and morphology, and a subsequent processing that may include various individual dehydration, clearing, infiltration, sectioning, and staining steps. Staining may include, for example, procedures for adding or removing paraffin, additional dehydration/hydration, staining, clearing, and preparing slide(s) that may have cover slips. These techniques are well known.

In an exemplary permanent-section process, a specimen is removed from the patient and sent to a pathology laboratory. At the lab, the specimen may be placed in a formaldehyde preserving agent and then be prepared for sectioning. It is sectioned into microscopically thin sheets, attached to glass slides, stained by various agents to enhance observation, and is then microscopically viewed by a pathologist who characterizes observed features and renders a pathology diagnosis. A processing may include any number of fixing, hydration/dehydration, and other associated steps.

In an exemplary frozen-section process, the specimen is often not preserved in a formaldehyde solution, but instead is taken directly to a freezing chamber. After freezing, it may be sectioned and mounted on a glass slide, after which it is stained and microscopically observed and diagnosed by the pathologist.

In both of these techniques, the specimen is stained, observed, and diagnosed after the tissue is first taken out of the patient and then processed.

Conventionally, an endoscope has been introduced into an existing body cavity through a bore in another device. Also, within an endoscope rod there may be an associated light source with a corresponding light channel and may also have other channels for introducing surgical instruments, water, air, or suction. Endoscopes optimized for various surgical procedures include arthroscopes, cystoscopes, proctoscopes, laparoscopes, and others. A typical endoscope, such as those embodying the present invention, may include an objective lens at its distal end for forming an optical image of the interior of a body cavity, bone, joint, or organ; may also include a transfer module (“relay”) for transmitting the image from the distal end to the proximal end of the endoscope; and may also include an ocular at the proximal end of the transfer module for presenting the image to an eyepiece, video camera, or other device or system(s). Such an ocular may include movable focusing apparatus. Exemplary contact endoscopes are described in U.S. Pat. Nos. 4,656,999 and 4,385,810, assigned to Karl Storz, each document being herein incorporated by reference in its entirety.

Another endoscope may utilize a Hopkins rod lens system having a greater amount of glass in its optical system, thereby providing a better medium than air for transmitting images, better light transmissibility, and a wider field of view. Rigid endoscopes may typically be formed with diameters from about one to ten mm, and will vary according to viewing angle, depth of field, magnification, image brightness, image quality and contrast, distortion, and image size. Additional uses with the present invention may include flexible endoscopes, including those utilizing fiber optics.

More recently, a “contact endoscope” has been made commercially available by the K. Storz Medical Company (Tuttlingen, Germany). Such a contact endoscope is formed as a rigid endoscope having a moveable lens within it. Such lens may affect a variable and/or switchable focus/power, and may be a part of a larger optical system that includes any of an objective, eyepiece, external viewing apparatus, and other optics. One design of such a contact endoscope is for viewing nasal tissue to observe and examine cells on the nasal mucous membranes of a patient. In this nasal examination, a cotton swab is first used to apply methylene blue stain directly to the patient's nasal mucous membrane. Thereafter, the contact endoscopic is placed into the nose. The endoscope is visually guided macroscopically to the area of staining until the distal end of the endoscope makes contact with the mucous membrane. Thereafter, using a focus knob on the side of the endoscope, the moveable lens is made to enlarge and focus onto the cells at a microscopic level. Such focusing may be in combination with other lenses of the optical system. Thereafter, the medical practitioner prepares her diagnosis of the mucous membrane based on the observed stained area.

This contact endoscope and staining method have generally only been used for accessing surface membranes of the nose, for diagnosing allergic rhinitis of the nose. Very recently, by comparison, gastric mucosa have been examined using a confocal endomicroscopy system (Y. Kakeji, et al.). Other outer surfaces such as the skin or cervix might be accessed by using a technique similar to the technique employed on the nose, but stain has not generally been used for such examinations.

The just-described Storz contact endoscope has no working channels and is not designed for penetration into the body, thereby only being appropriate for contact with surface structures. Such a contact endoscope cannot access or penetrate into deeper tissues because it has a slim, fragile optical rod and a flat non-streamlined lens tip. Such a conventional contact endoscope entering into the body and pushing to deeper target tissues would not be not practical because such would cause damage to the endoscope and to the body tissues. Additionally, such tissues, even if endoscopically accessed, would not be stained. By staining cells, multiple cellular characteristics are enhanced so that the cell types become apparent for histology diagnosis. Without staining, the human eye, even with microscopic magnification, cannot distinguish the various cellular outlines and internal cell structures of in vivo tissue. It is noted that conventional contact endoscopy has included limited direct viewing of body surface pathological tissue, but has not included the devices and methods of the present invention allowing for a complete body, multiple tissue staining, in-vivo pathology diagnosis.

There is a need for an endoscope system and for methods that allow for staining tissues, for accurate endoscopic placement in a patient's tissues including deep tissue, for deposit of chemicals or materials, and for making a histologic diagnosis in-vivo enabled by the equipment and methods.

Known endoscopes may have 0°, 25°, 30°, 45°, 70°, and 120° angles at their viewing tips proximate a distal end. These angles specifically allow the observer to see from a position at the end of the endoscope in non-forward directions. For example, if the user inserts such an angled lens endoscope straight into the nasal cavity, the angled lens would permit the user to see the side walls of the nose. Such conventional endoscope tips, specifically those that are angled, are blunt and are not typically designed or intended to push through body tissues. These endoscopes are instead designed to view objects through air and are not designed to contact deep body tissues. They typically have a fish-eye view and are meant for macroscopic, large fields of view. Indeed, a view of tissue using air type endoscopes will become completely blurred if such devices are used for directly contacting tissues.

It is noted that some conventional endoscopes are used in conjunction with a separate trocar device or biopsy punch to push through tissues, such as the front of the cheek sinus that has a thin bone wall.

Definitions:

“Pathology” as used herein is the study and diagnosis of disease and diseased tissue through examination of organs, tissues, cells, and bodily fluids.

“Endoscope” as used herein refers to an elongated optical probe capable of presenting a visible image of the interior of a body tissue to a physician via a viewing device such as an eyepiece or video screen. It is noted that certain endoscopes are highly specialized, such as those for colonoscopy which are inappropriate for uses such as surgical examination that requires penetration of tissue. Similarly, conventional use of an invasive catheter to help inspect heart tissue through cardiac catherization is unrelated to an endoscope of the present invention.

“Endoscope sheath” as used herein is a protective tube-like apparatus for being inserted into a patient and then receiving a rigid endoscope and protecting the endoscope from damage. As used herein, the term may also be referred to as a “cannula”.

“Contact endoscopy” uses an endoscope having a front lens that may include an objective lens assembly and/or a cover plate disposed at or proximate the distal end of the endoscope, where the front lens or distal cover plate is brought into contact with a target tissue to be observed.

“Optical forceps” integrate a protective sheath and biopsy or retrieval forceps into a single instrument that may lock onto an endoscope or other instrument.

“In vivo” as used herein is defined as being in the living body of an animal.

The terms “stain” and “staining” as used herein pertain to in vivo histotechnology processing.

Additional definitions are listed in U.S. Pat. No. 7,183,381, incorporated herein by reference in its entirety.

### III. SUMMARY OF THE INVENTION

Devices and methods described herein allow a physician to access deep tissues and make corresponding pathological diagnoses, affecting real-time surgery. It will be known as “in vivo pathology diagnosis (IVPD)”. Such a use may be in conjunction with a contact endoscope, or with a conventional endoscope (in-air usage) within an endoscope sheath containing a tissue contact lens at its tip.

By use of the devices and methods, improved diagnosis of pathologic medical conditions is based on improved access, inspection, and modification of tissue deep below the surface layer. The devices and methods of the present invention may also be applied for in vivo histology examination not limited to pathology diagnosis, but for finding targeted normal tissues.

According to one aspect of the present invention, materials may be placed at a destination location within tissue. For example, staining fluids may be placed for precisely staining certain target tissue in preparation for pathologic diagnosis. By using multiple endoscope working channels one or more irrigation-suction streams may be created.

According to another aspect of the invention, an endoscope is provided that enables pushing the endoscope to deeper target tissue areas. For example, an improved endoscope, or a sheath for use with conventional endoscopes, is provided that allows for streamlined or cutting advancement through tissues. This can give clear visual inspection of a deep tissue target such as by use of multiple magnification lens assemblies.

According to another aspect, a chamber is formed at a distal end of the endoscope or sheath, the chamber being in fluid communication with one or more channels for precise control of in vivo staining. According to a further aspect of the invention, such precise control is increased by use of additional apparatus and/or channels. Such uses may include titration, flow rate and pressure control, rinsing, microprocessor control, feedback systems, etc.

It is understood that enhanced visualization and access to deep tissue with minimal trauma is desirable. Improvements in accuracy resulting from recent advances in optics available for endoscopy provide reduced postoperative pain and accelerated recovery. Embodiments of the present invention are intended to embody these advantages, for example including cauterizing in some applications. Rigid endoscopes may provide additional advantages compared with flexible devices because of improved optics and reduced costs.

These and other features will become apparent upon review of the drawings and detailed description, presented below, which set forth the preferred embodiments of practicing the invention perceived presently by the Applicant. The foregoing summary does not limit the invention, which is defined by the attached claims. Similarly, neither the Title nor the Abstract is to be taken as limiting in any way the scope of the disclosed invention.

### IV. BRIEF DESCRIPTION OF THE DRAWING FIGURES

FIG. 1A is a schematic illustration of a sheath device enclosing an endoscope that is longitudinally movable within the sheath, according to an exemplary embodiment of the invention.

FIG. 1B is an enlarged view of the distal end of the sheath and endoscope of FIG. 1A, illustrating features that include a front lens of an endoscope and tip openings of channels of the sheath.

FIGS. 2A and 2B are schematic illustrations of two exemplary variations of a segmented endoscope tip lens each having multiple viewing and magnification segments, the segments having different focal lengths, curvatures, magnifications, etc., for simultaneous viewing and other applications.

FIGS. 3A and 3B respectively schematically illustrate an endoscope system in an extended and retracted position, where an endoscope tip lens has a rounded lens surface that may serve as more than one viewing and magnification area simultaneously, according to an exemplary embodiment of the invention.

FIGS. 4A-4B respectively schematically show a penetrating sheath device having a contact endoscope in extended and a retracted positions, the contact endoscope and surrounding sheath each having a pointed/slanted tip, according to an exemplary embodiment of the invention.

FIGS. 5A-5C schematically show an exemplary embodiment of a penetrating sheath enclosing an endoscope that may have a penetrating tip, the sheath having openings along sides of a tip area that act as portals to allow body tissues to move inwardly toward a lens at the tip of the endoscope.

FIGS. 6A-6B schematically show exemplary embodiments of a penetrating endoscope having a sharp leading edge that allows it to slide into and through tissues, FIG. 6B showing an exemplary embodiment where the sharp leading edge is formed as a detachable attachment.

FIGS. 7A-7E show exemplary embodiments of an endoscopy cauterizing system that allows cauterizing tissue in a same procedure involving tissue penetration and/or staining, where FIGS. 7A and 7B respectively schematically show a monopolar type cautery system and a bipolar type cautery system, FIGS. 7C and 7D respectively schematically show both type systems in an endoscope sheath and in an endoscope, and where FIG. 7E schematically shows a cautery rod system that may be placed through an endoscope working channel.

FIGS. 8A-8B schematically show an exemplary embodiment of a penetrating endoscope having an integrated staining system and/or cauterization system.

FIGS. 9A-9D, schematically show an exemplary embodiment of a penetrating endoscope having a working channel.

FIG. 10 schematically shows an exemplary embodiment of a penetrating endoscope having a rounded lens tip.

FIGS. 11A-11B schematically show an exemplary embodiment of a partial sheath having a staining system, working channel(s), and a cauterizing system.

FIG. 12 schematically illustrates an exemplary stepped type lens tip that may be used in suitable embodiments of a penetrating endoscope system with a multiple-magnification tip.

FIGS. 13A-13C schematically show a bone penetration type endoscope system that includes a sheath having a structure for enclosing either a drill bit or an endoscope, according to an exemplary embodiment of the invention.

FIGS. 14A-14D schematically show an exemplary embodiment of a retractable tip that may be adapted for use with either a penetrating endoscope or a penetrating sheath.

FIG. 15 schematically shows an endoscope or sheath shape that uses an overall cork-screw form adapted to enable tissue penetration, according to an exemplary embodiment of the invention.

FIGS. 16A-16D schematically show an exemplary embodiment that may be implemented in either a penetrating sheath or a penetrating endoscope and that has a structure adapted for insufflation of air, a specific gas, or a liquid, a tissue stain or a gel through respective insufflation and deflation channels.

FIG. 17A schematically shows a conventional endoscope and FIG. 17B schematically shows an exemplary embodiment of an attachment to a conventional endoscope that transforms it into a microscopic IVPD instrument.

FIG. 18 schematically shows an exemplary embodiment of control apparatus used for precise control of operations related to use of an endoscopy system of the invention.

FIG. 19 is a schematic illustration of an alternate embodiment sheath device enclosing a conventional endoscope that is longitudinally (axially) moveable within a tissue-contacting lens containing sheath, according to an exemplary embodiment of the present invention.

### V. DETAILED DESCRIPTION OF PREFERRED EMBODIMENTS

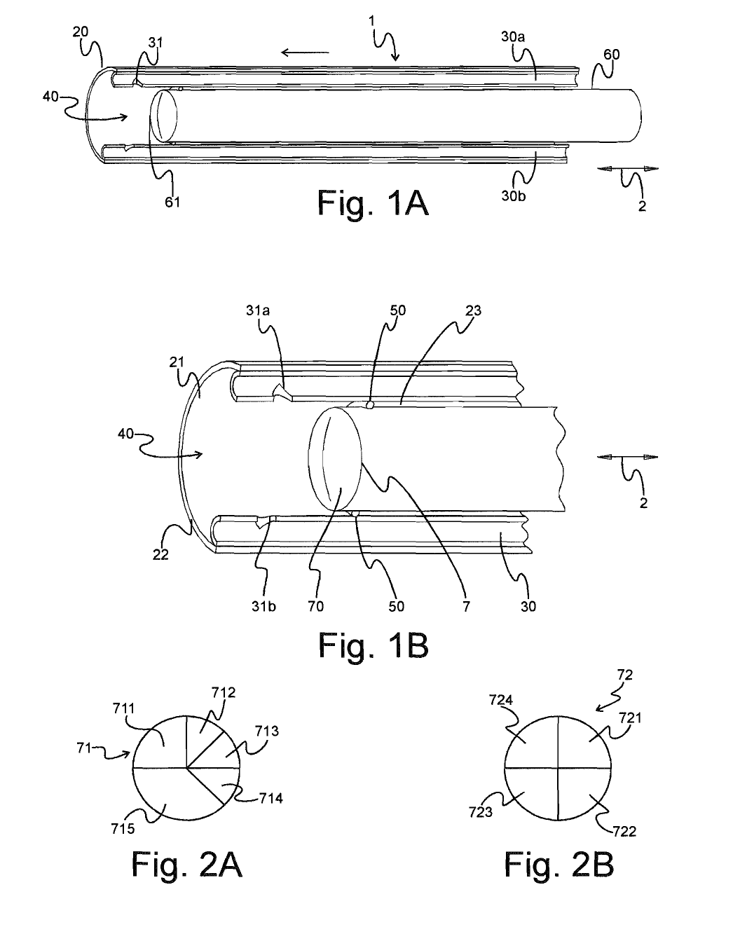

FIGS. 1 and 1B schematically show an endoscope system 1 having a sheath 20 (illustrated in cross section) adapted for protecting a contact endoscope 60 within a cylindrical inner space 21. Contact endoscope 60 is longitudinally movable within sheath 20, which effects a protective sleeve. Sheath 20 contains one or more irrigation channels 30 that are preferably in a same longitudinal direction 2 of movement of contact endoscope 60. Channels 30 are used to flush staining fluids into and across body tissues of interest in order to stain the tissue cells (not shown) in preparation for pathological diagnosis. A single channel may be used to first flush and then remove the stains. In a preferred embodiment, at least two or more channels 30 are used. With such a use of two or more channels, a continuous irrigation-suction stream of one or more stains can be used. Stains may thereby flow over the tissues sequentially or simultaneously, by entering an inflow port (not shown) of sheath 20 that is exterior to the body being examined, then flowing down an inflow channel 30a, and then exiting from an opening 31 at the tip area 22 of sheath 20. After exiting from opening(s) 31a from a stain inflow channel 30a, the stain will touch body tissues and stain the tissues. A chamber 40 is formed in a space between a distal end 61 of contact endoscope 60 and sheath tip 22. A stain exit opening 31a injects the stain into such chamber 40 and a stain retrieval suction opening 31b receives such stain after it has touched body tissues within an area proximate chamber 40, such retrieval being effected by a use of suction applied to a corresponding suction channel 30b.

Chamber space 40 is designed to permit a chamber effect whereby the stain irrigation is controllably contained to the site of pathology examination proximate the endoscope tip 61. By controlling the area (footprint) and volume of chamber 40, the stain flow rate, the stain concentration, the flow and quantities of stain and washing solutions and other materials, the suction flow, and the interactive effects related to time rates of fluid change and toxicity and other effects, such controllable containment will limit take-up at the target tissue and associated take-up by the blood. Such volume may be controllably varied by movement of contact endoscope 60 in direction 2. It is noted that ancillary surgical procedures may be used to further assist containment, for example by temporary vascular restriction. The design shape and structure of chamber 40 is also important because it limits the tissue staining process to the area directly in view by contact endoscope 60, for example by matching a contour of a sheath end with that of target tissue and/or by forming a stain barrier of a chamber wall that directs stain or other fluid flow.

Chamber 40 thereby prevents or greatly reduces extravasation and leakage of stains into adjacent or distant body tissues and limits the staining of tissue to the viewing area. However, surgery invading highly vascular tissue necessarily will present challenges in localizing staining materials. However, by limiting the inflow times to when a chamber 40 is securely abutting or enclosing target tissue, prior or subsequent invasion of vascular tissue may be separately addressed by other methods. For example, in U.S. Pat. No. 5,956,130, incorporated herein by reference, real-time knowledge of the rate of blood loss allows adjustment of intravenous fluid administration to maintain hemodynamic stability and for other fluid controls. In our case, when a penetrating device is being directed to the target tissue, ancillary fluid monitoring, especially related to blood loss, allows a stable target space to then be prepared for staining.

An endoscope system 1 with an inner endoscope may use a seal, such as an O-ring 50, to help contain the stains to the chamber 40 area and prevent backflow retrograde between sheath 20 and an interior adjacent space 23 within, such space circumferentially surrounding contact endoscope 60 as it slides within sheath 20. Interior space 23 may be pressurized according to any known method, for assuring and testing the integrity of space 23, for example to prevent contamination such as mixing of fluids, damage to endoscope 60, and other related problems.

Either sheath 20 or contact endoscope 60 may be formed as a “penetrating” device that, itself, is able to penetrate into the body to reach deeper tissues, to stain deeper tissues, and achieve hemostasis on exiting the body. The present inventors have discovered that certain procedures may allow a single instrument with a penetrating tip to be used in place of conventional punches or biopsy cutting tools that are used before inserting a separate endoscope.

An optical system implemented in contact endoscope 60, for example, may include a lens 70 or system of lenses placed in front of an objective and having its external side(s) adapted for being placed in direct contact with the target to be examined. As shown in FIG. 1B, contact endoscope 60 has a rounded front lens 70. FIGS. 2A and 2B respectively schematically illustrate two exemplary segmented lenses 71, 72 that may be used as a front lens 70. With the rounded front lens 70, a penetration of tissue may be performed by use of a sharp tip (discussed below) of sheath 20. Subsequent steps may include staining of such target tissue, followed by a subsequent positioning of contact endoscope 60 for viewing stained target tissue.

FIG. 19 illustrates an alternate embodiment endoscope system 600 having a sheath 602 adopted for protecting a conventional endoscope 604. Endoscope 604 is longitudinally moveable within cylindrical inner space 606. Sheath 602 contains one or more irrigation channels 608 that preferably extend in an axial direction, along the same direction as the direction in which endoscope 604 moves. Preferably, sheath 602 endoscope 604 and irrigation channels 608 are all disposed co-axially.

Endoscope system 600 differs from other endoscopes of the present invention because endoscope 600 includes a tissue contacting lens 610 that is coupled to the distal end of the sheath 602, radially inwardly of the irrigation channels 608. This arrangement permits a conventional endoscope 604 to be used with the present invention, and enables the endoscope to gain an optical corrective diopter in order to facilitate visualization of tissue when the distal tip of the sheath touches tissue. Depending upon the particular corrective diopter and magnification chosen, the image visualized through the prior art endoscope 604 will be a clear, magnified visualization of the tissue of interest.

Parts of cells and intercellular material are usually transparent and, accordingly, stains are conventionally used for pathology diagnoses of tissue that has already been removed by a biopsy procedure. Stains are typically high in purity, and are diluted to an appropriate level for use in differentiation between different types of tissue when applied thereto.

Various staining procedures may be optimized for particular target tissue, for example when staining collagen or elastin using deposits of metal salts. Thiazins such as methylene blue are synthetic dyes that may be used for staining both living and fixed tissue such as pathologic tissue, for making various tissue and cell constituents more evident. Certain types of staining, for example intravital staining, depend on dye uptake by phagocytic cells, and may be prepared as a colloidal solution of nontoxic coloring matter. The size of individual dye particles is considered for the application.

A control of staining process may include testing of the target tissue for pH and other indications of how the stain(s) will interact with the target tissue and surrounding cells. Another factor is the variability and non-homogenous nature of tissues. For example, some tissues will have more available amino groups than others and will therefore likely attract different quantities of an acid dye based on degrees of basicness. Many other factors will determine which particular staining material(s) are used. Examples of such factors include the degree to which carboxyl groups reach the amino groups that will attach, the dynamic nature of individual events that result in a particular chemical reaction, reversibility of chemical reactions and elapsed time for a given reaction event compared with a corresponding completion time (e.g., equilibrium) for the event or series of events, mass action, interactions, quantities of individual reactants involved and presence of products, interruption of process such as by removal of one or more reactants or products, solubility, etc.

In histological staining, a dye and its components react with tissue groups and with any dyes already attached to such tissue groups. Histological staining may bias this process toward one individual component at a time by applying relatively strong dye solution(s) for a limited time, then removing them, and then applying other solutions in a series of timed biased events that prevent equilibrium from being reached in selected solutions or that allow some equilibrium for strictly limited times. Such processes typically remove dye when the degree of staining is achieved.

An advantage of the present invention is simultaneous staining and visual observation of the degree of staining, for improved accuracy. Ancillary steps such as stain removal and washing are also more accurate with real-time visual control. The aforementioned equilibrium may be considered gross overstaining, but is reversible by procedures such as washing a slightly overstained tissue with water, thereby reducing an original reactant and biasing the reaction against it, resulting, for example, in a reduction in the depth of staining. Many other examples of controlling dynamic steps of the staining processes, and corresponding complex results, are achieved as a result of the disclosed in vivo staining system.

Suitable dyes in a thiazin class include methylene blue, chosen for its application as a non-toxic dye that stains nuclei blue, ripens, and produces three strongly metachromatic dyes, Azure A, B and C. Such solution is commonly referred to as polychrome methylene blue and is valued for demonstration of mucins, cartilage, mast cells, etc. Sensitivity and specificity for strong staining allows detection of pathologic tissue. It is noted that recently, Olliver et al. reported (See, e.g., Sidorenko et al., “High-resolution chromoendoscopy in the esophagus,” Gastrointestinal Endoscopy Clinics of North America, Vol. 14, Issue 3, p. 437-451) that exposure of certain mucosa to methylene blue and endoscopic white light can lead to DNA damage; therefore, precision control of dye exposure time is important in limiting toxicity. However, the additional feedback of visual inspection of in vivo staining, and the simultaneous real-time pathology diagnoses achieve many results not seen in conventional methods such as surgical procedures performed in conjunction with fluoroscopic methods, and may offer different staining controls and dynamics of perfusion compared with conventional extravasation and the like.

Methylene blue has a relatively poor penetration in deeper layers, but such helps limit staining to the target area. The tradeoff between poorly described infiltration and high staining controllability may make it difficult to decide whether an image is sufficiently diagnostic. Therefore, close collaboration with a pathologist is necessary. After a staining period, the stain may be extracted using graded strengths of ethyl alcohol and distilled water rinse, and the tissue may be analyzed with a spectrophotometer, for example in a range of 500-900 nm, and preferably at about 660 nm (Abs 660 nm). To reduce toxicity, a number of wash cycles may be used, for example with water. A recent example of accuracy of methylene blue staining in vivo as published by Canto, et al. is “Methylene blue staining of dyplastic and nondyplastic Barrett's esophagus: an in vivo and ex vivo study,” Endoscopy, 2001 May; 33(5): 391-400.

Supravital staining involves the application of specific dyes that penetrate all cells and color certain cellular or tissue components. For example, methylene blue (0.025% to 0.25%) has been used to demonstrate nerve endings in muscle tissue. In hematology, supravital staining with solutions of Janus green B and neutral red assist in distinguishing myeloblastic from lymphoblastic leukemia.

A dye used in supravital staining must enter the cell and also diffuse through the protoplasm without killing the cell, and must color preexistent cell inclusions distinctively or color the whole of the cytoplasm of particular cells strongly enough that those cells stand out from intercellular material and other cells. As a result, the nucleus and ground cytoplasm are affected very slightly, but cytoplasmic inclusions such as vacuoles, lipid globules, mitochondria, and others are colored. Such dye has difficulty entering the nuclear membrane and the phosphoric groups of the DNA are still combined with protein and are not free to react with the basic dye if the dye is not permitted to remain in the target tissue. By subsequently washing and changing the reactions, excess dye is removed. However, in vital dyeing, the concentration at which a dye will act cannot be controlled, and a state of equilibrium is built up between the dye and the fluid of the target cell, so that differentiation, washing and suction are necessary for controlling unwanted distributions of stain.

An objective of differentiation is for desired features to retain sufficient stain to be visible and for other tissue components to be cleared of dye. Alcoholic solutions may provide better results compared with the aqueous, and 95% or absolute alcohol may be used as a stock solution. Water is used for dilution, and a water wash will remove excess differentiating fluid. In addition, while exact mechanisms of metachromatic staining are relatively undefined, the absorption spectra of aqueous solutions of metachromatic dyes change with variations in concentration, pH, temperature, and others, so that monitoring of such parameters helps optimize an in vivo staining process. For example, in relation to an increase in dye concentration, an alpha peak corresponds to monomer dye molecules in dilute dye, a beta peak corresponds to formation of dimmer molecules as concentration increases, and a gamma peak observed in metachromasia is attributable to formation of polymer dye molecules in tissue.

It is noted that water molecules intercalate between the dye molecules and, therefore, have an influence on metachromatic reaction(s). Conversely, treatment with a dehydrating agent such as alcohol completely destroys the metachromatic reaction. In many applications, it is important that the methylene blue stain be pure, so it is preferable that bursts or pulses of dye be input to chamber 40 rather than inputting an already diluted material, and it is also preferable that stain be highly localized to the target tissue.

Additional influencing effects include physical aspects such as surface area and density of the absorbing tissue, the size of the adsorbed particles, and chemical factors. Further, tautomeric forms of a same dye may have different chemical and physical properties that cause them to be adsorbed differently. Still further, the pH of dye, differentiating agent, tissue, and other components all influence uptake of dye by tissue and its subsequent removal. Cell nuclei, for example, are acidic in character because of nucleic acid components and will stain with basic dyes such as methylene blue. However, cytoplasm is comparatively basic and will stain with acid dyes such as eosin type material. Methylene blue stain may be also prepared as an acid, alkaline, polychrome, metachrome, and other.

By controlling inflow and suction, a precise amount of dye is applied to chamber 40 when tip 22 is securely abutting or enclosing target tissue, where different stains require corresponding time periods of contact with the tissues to attain the desired degree of perfusion. After the stain(s) are exposed to the tissues, they exit the tissue site by being suctioned up through an exit opening 31b in tip 22 of the device 1. Exit opening 31b in such a case draws the staining or other material into an outflow channel 30b, and such material exits channel 30b at a proximal end (not shown) of device 1, for example via a fluid output of a suction pumping device (not shown). Because staining materials are foreign substances, they trigger defense mechanisms that may cause different reactions compared with ex vivo pathology. However, such defense mechanisms may also limit perfusion and may also limit unwanted capillary distribution of materials, for example when a dye is consumed by defense cells resulting in localization of staining materials.

By creating a continuous inflow and suction, and by utilizing inflow channels 30a for materials such as water washes, the degree of staining is controlled and a clear site exists for visual observation using contact endoscope 60. Peripheral external equipment may include titration devices and injectors, manifold(s), bulk supplies, filtering, spectrophotometric equipment, detectors, analyzers, ionization devices, pneumatic devices, and others. By way of example, a computer controlled staining process may include the injection of precise pulses of dye into an irrigation channel 30a, with time delays and multiple pulses of water wash between stain pulses. In such a case, individual pulses of other material 700 may, for example, include acid at a very low concentration followed by another series of water wash pulses, while maintaining a continuous suction through suction channel 30b. Various metachromatic results as viewed through contact endoscope 60 allow the user to dynamically modify such procedures using feedback information.

As discussed further below, computer algorithms may assist in monitoring dyes and proteins involved in histological staining including chemical bonding processes, such as by monitoring color of materials 700 being input and those being suctioned, blood analysis, as well as real-time visual analysis of the target area and surrounding tissue. Dyes are easily manipulated, and corresponding detected color allows identification of individual components of tissue areas/sections. It is noted again that dyes and associated materials are possibly toxic, for example carcinogenic or mutagenic or otherwise harmful, but accurate control of exposure times, ionization, etc., may greatly reduce such toxic effects. Additional procedures may also reduce toxicity, such as counterstaining, vascular restriction, etc. Further, it is important that the effects of the staining itself be scrutinized for reducing additional disease and injury to the target tissue area and for accuracy of diagnosis. For example, staining may induce cellular adaptation to injury, cell death, inflammation, tissue repair, neoplasia, or other.

After the staining process is complete, contact endoscope 60 is advanced forwards through sheath 20 to a point where front lens 70 is in contact with the tissues to permit the user to visualize the cells. An inflow of an additional material may assist in improving the clarity of fluid surrounding the target tissue, such as by replacing blood surrounding such tissue with solution that is at least partially transparent, for example improving observation by compensating for macroscopic motion by excluding changes caused by blood flow, by suctioning such blood via exit channel 30b and replacing it with a transparent solution immediately before viewing the target tissue.

Chamber 40, shown schematically in FIGS. 1A and 1B, allows staining in methods of “In Vivo Pathology Diagnosis” (IVPD). The stain may be placed by eye-hand coordination, with the user deciding when enough stain has been taken up by the tissues. Alternatively, the sequence of stains may be placed in a programmed amount by a single or multiple series of pumps connected to sheath 20 or endoscope 60. Such device is preferably respectively adapted to be used either for deep tissue or at the surface. When tip area 22 of sheath 20 is located at the target, additional uses of the corresponding inflow channel(s) 30a, chamber(s) 40, and outflow channel(s) 30b include delivery of medications, chemicals and compounds, slow-release materials, nucleotides, viral and bacterial carriers, radioactive materials, micro-capsules or containers, clotting and anti-coagulant agents, and other substances.

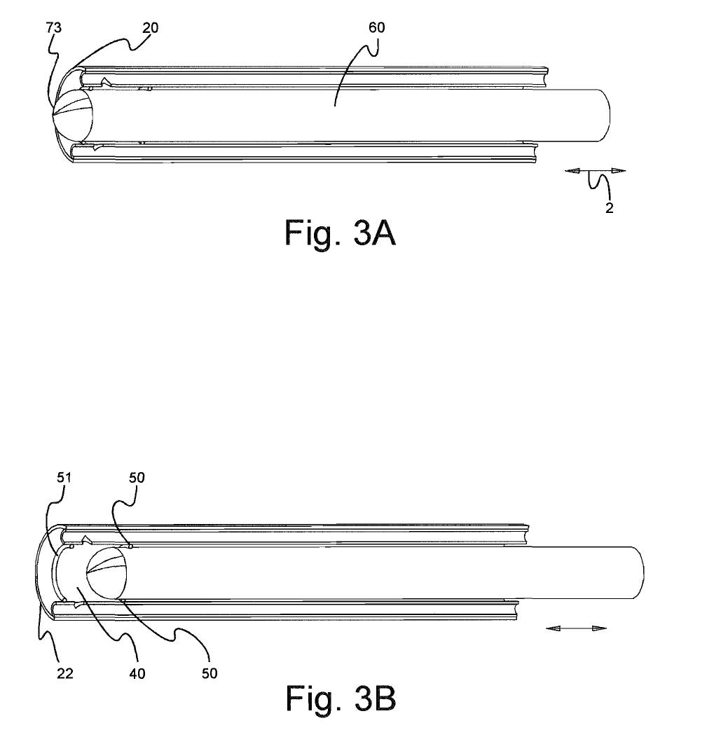

Endoscope tip lens 73 as schematically shown in FIGS. 3A-3B has a rounded lens surface that may serve as more than one viewing and magnification area simultaneously. In such a case, different lens curvatures and magnifications are affected by individual segments positioned in different parts of endoscopic tip 7. For example, FIG. 2A schematically illustrates a lens tip 71 that can serve as one or more lens magnifiers simultaneously. Thus, segment 711 of lens 71 may be dedicated to visual magnification power 1, another segment 712 to power 2, another segment 713 to power 3, etc. The example of FIG. 2B schematically illustrates a lens 72 having segments 721, 722, 723, 724 each having a same shape, with magnifications that are preferably different, for example allowing different preset focal lengths to be selected. Endoscope 60 may be preset to be focused upon tissue contact without the need for a focus knob. Alternatively, a magnification tip may employ a conventional contact endoscope lens assembly having a moveable lens focus knob.

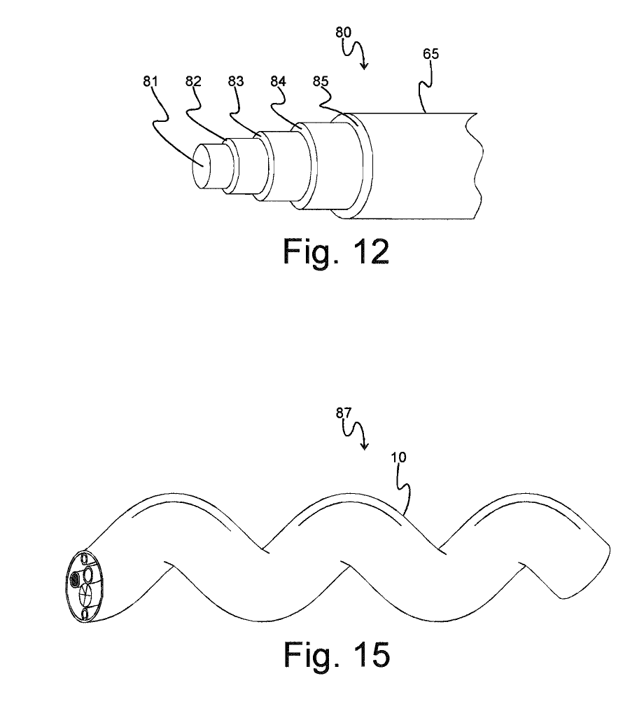

FIG. 12 schematically illustrates a stepped type lens tip 80 that may be used in suitable embodiments of a penetrating endoscope system to affect another form of a multiple-magnification tip endoscope 65. Lens tip 80, for example, has a central core that projects to a distal end as a lens surface 81, and has additional concentrically disposed lens surfaces 82, 83, 84, 85 in a stepped arrangement. Individual lens surfaces 81-85 may have either flat shapes or be contoured as rounded shapes or edged shapes, with streamlined or sharp profiles. Each of the flattened step-offs 81-85 is preferably a lens adapted/preset to focus at a different power on the target. Each step-off 81-85 may have one or more magnification powers, for example by segmenting.

Additional embodiments address other particular problems encountered with a contact endoscope 60 and the penetrating sheath-type devices 20 and methods of IVPD, and will be individually explained.

FIGS. 3A-3B show a rounded tip 73 of an endoscope 60. Such endoscope 60 sits within sheath 20 and is moveable within and removable there from. This rounded tip example of endoscope 60 may be formed to be much more streamlined than an endoscope having a blunt tipped tip area 7. For some applications, such streamlined rounded tip 73 allows endoscope 60 to easily pass and penetrate into and through body tissues. For example, rounded tip 73 may serve as the leading and penetrating edge of a penetrating device 1, having less penetration resistance compared with a flat tip.

To enter deeper tissues, endoscope tip 73 is placed to a position just past sheath tip 22 to create a rounded tip configuration for the combined endoscope sheath unit as shown in FIG. 3A. Once the desired location is reached, endoscope 60 is retracted to create a chamber space 40, as shown in FIG. 3B. A sliding motion 2 of endoscope 60 relative to sheath 20 acts to form a seal in the extended position, as a result of O-rings 50, 51, thereby reducing contamination of the inside space 23 and contact endoscope 60. Tip 73 is made of a transparent lens material and may have one or more focus or magnification factors as segments. A locking member (not shown) is preferably used for securing endoscope 60 at a fixed position relative to sheath 20, for example holding endoscope 60 at a retracted position during staining or holding endoscope 60 at an extended position during tissue viewing and diagnosis.

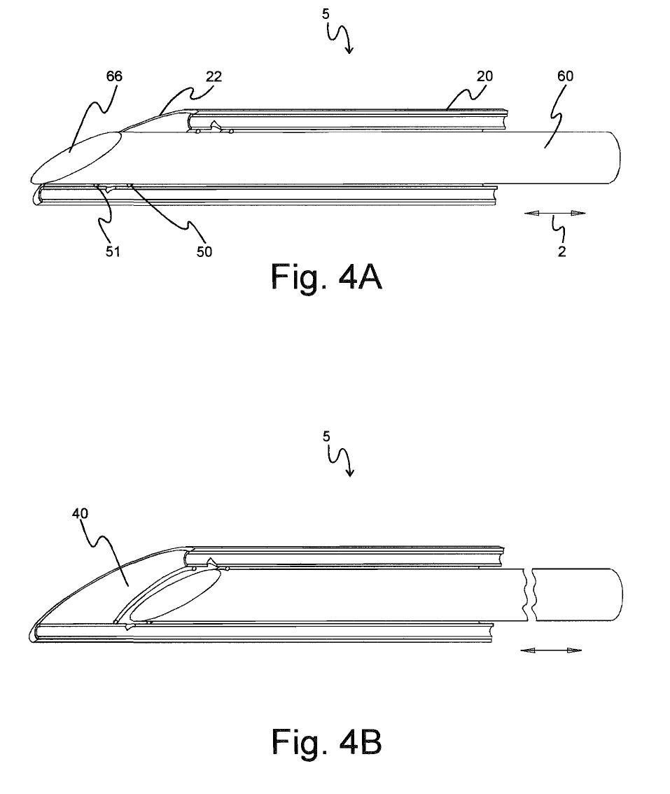

FIGS. 4A-4B respectively schematically show a penetrating sheath device 5 in an extended and a retracted position. Contact endoscope 60 has a pointed/slanted tip 66 and a surrounding sheath 20 has a pointed/slanted tip 26. Pointed tip 66 may have its apex either at a centered or off-center position as shown. Tip 66 preferably has a very sharp apex for relative ease of puncturing to reach target tissue. In this example, sheath tip 26 is open for passage of contact endoscope 60 there through. A slanting angle of sheath tip 26 is preferably the same as for endoscope tip 66, for example allowing a full view when guiding sheath 20 or for reducing the width of a resultant incision when tip 66 is the cutting edge. As in other embodiments, endoscope tip 66 may be transparent and have several facets or segments, each having a different magnification.

The open end of this sheath 20 is closed by the sliding action of the angulated endoscope 60, or opened for creating chamber 40. In the closed position, the streamlined tip may be more easily advanced through tissues while preventing contamination of a chamber 40 space. Once the desired area is reached, endoscope 60 is retracted away from the distal end 22 of sheath 20 to create a chamber area 40, into which tissue staining material(s) or other material(s) are introduced. A method of In Vivo Pathology Diagnosis (IVPD) may subsequently be performed.

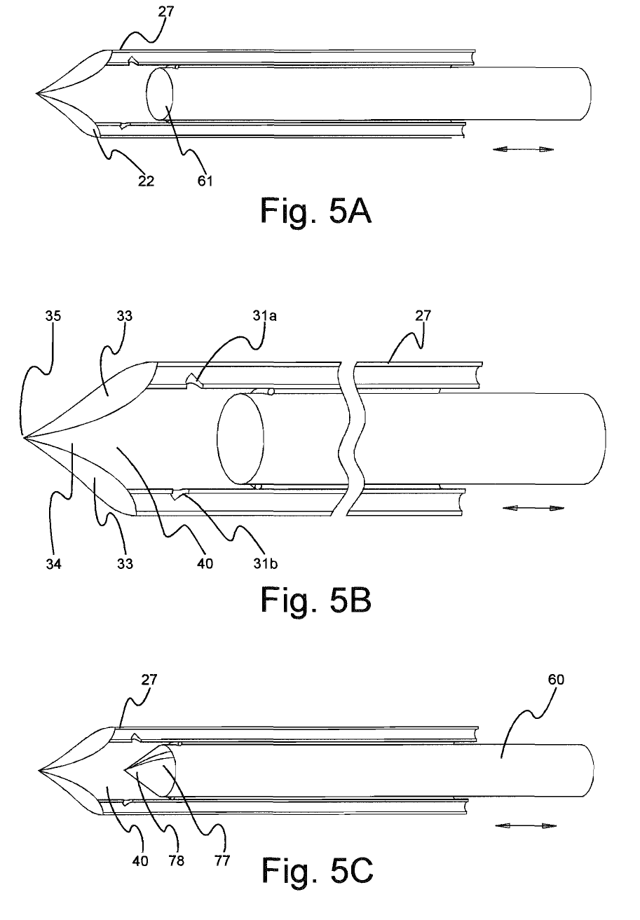

FIGS. 5A and 5B respectively schematically show a penetrating sheath 27. FIG. 5C schematically shows a penetrating sheath 27 surrounding an endoscope 60 having a penetrating tip 67. Sheath 27 has openings 33 along sides of a tip area 34 creating portals that allow body tissues to herniate inwardly toward a lens at distal end 61 of contact endoscope 60. As shown in FIG. 5C, when a penetrating lens 77 is behind the pointed, leading sheath edge 35 and tip 34, a chamber 40 is formed for tissue staining purposes. The herniated body tissues entering chamber 40 are stained and visualized with endoscope 60. With this sharp sheath edge 35, sharp tip 34 may assist penetration into tissues, while opening portals 33 are designed for defining the contact of body tissues with lens area 61, for example with lens 77. A tip of endoscope 60 may be a pointed lens tip 78 or a flat tip 61.

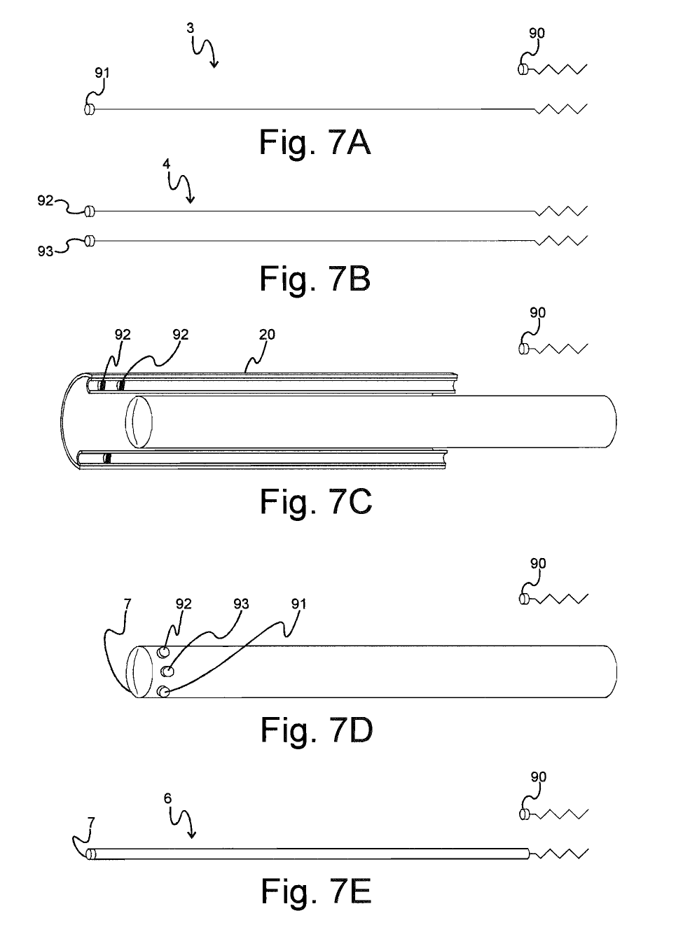

A given embodiment of the invention may be adapted for cauterizing tissue in the same procedure involving penetration and/or staining. For example, cauterizing may control blood vessels that are bleeding, which may then permit a higher accuracy of staining when bleeding is arrested. FIGS. 7A and 7B respectively schematically show a monopolar cautery system 3 and a bipolar cautery system 4. Either or both of cautery system 3 or 4 is attached to or within the sheath device 20. Either or both of cautery system 3 or 4 may also be a structure in or on an endoscope 60. In an exemplary embodiment, cautery systems 3, 4 cauterize bleeding vessels during entry of penetrating sheath 27 or endoscope 60 into the body, and especially prior to exiting the body. With penetration, vessel damage and bleeding may occur and it is important for the user to have a means of stopping any bleeding problems.

Monopolar electrode system 3 includes a monopolar electrode 91 having a distant ground 90. Bipolar electrode system 4 includes a pair of electrodes 92, 93. FIG. 7C schematically shows both a monopolar electrode system 3 and a bipolar electrode system 4 as components of a sheath 20. FIG. 7D schematically shows electrode systems 3, 4 being components of an endoscope 60.

In a further embodiment, as shown in FIG. 7E, a “cautery rod” system 6 may have either or both of monopolar electrodes 3 or bipolar electrodes 4 and may be placed through an endoscope working channel 30 to achieve bleeding control at the endoscope tip 7. Monopolar system 3, bipolar system 4, and cautery rod system 6 are preferably embodied in devices that derive their energy from an external power source.

FIGS. 11A-11B schematically show an embodiment of a partial sheath 46 having a staining system with channels 30 and having a cauterizing system with electrodes 92, 93. Partial sheath 46 is attached to the external surface of contact endoscope 60 by an attachment member 56. For example, partial sheath 46 may be formed to enclose only a circumferential portion of endoscope 60 and be held thereto by use of clips, loops, fasteners, and/or guide rails. The length of the staining and cautery systems may extend along an entire endoscope length, or have a different length. For example, a shorter version may be used when less depth is required while examining surface tissues. Sheath 46 is well-suited for retro-fitting existing endoscopes to upgrade them for use in In Vivo Pathology Diagnosis. Additional working channels 36 are shown for introduction of instruments, laser fibers and other apparatus.

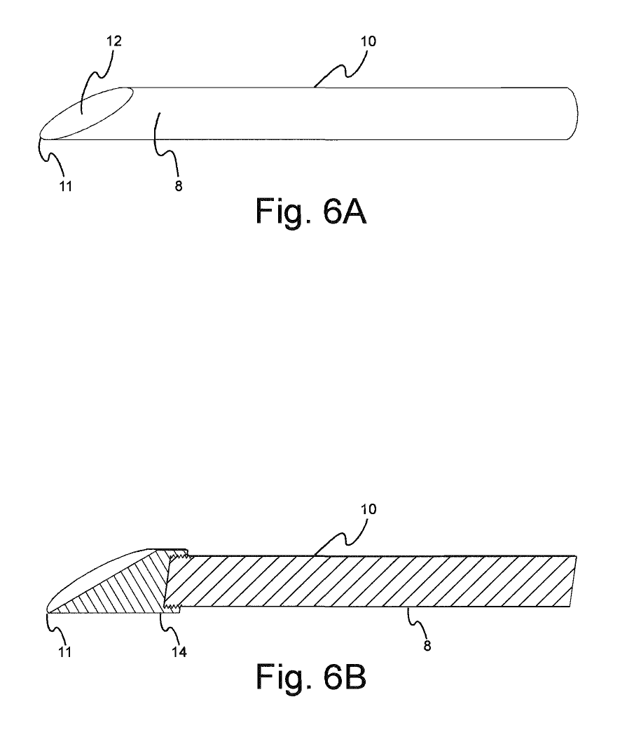

The exemplary apparatus shown in FIGS. 6A-6B, 7A-7E, 9A-9D, 10, 12, and 15 may be embodied for use in In Vivo Pathology Diagnosis (IVPD) as actual parts of an endoscope, and not as part of a sheathing device fitting over the endoscope. As used herein, such an endoscope may be referred to as a “penetrating endoscope.”

FIGS. 6A-6B schematically show a penetrating endoscope 10 having a sharp leading edge 11 at the tip 12. Leading edge 11 allows penetrating endoscope 10 to slide into and through tissues much more efficiently compared with conventional endoscopes. FIG. 6A shows an endoscope shaft 8 having an integral, permanent pointed tip 12 having a sharpened leading edge 11. As shown by example in FIG. 6B, the sharp leading edge 11 may alternatively be detachable through the use of an attachment member 14 secured to the endoscope shaft 8. An advantage of a detachable sharp edged tip embodied in a removable attachment 14 is that it can be exchanged for a new sharp edge if the edge 11 becomes dulled. Examples of attachment mechanisms used for attachment member 14 having sharp leading edge 11 are a screw-on mechanism as shown, various clip-on mechanisms and/or an adhesive mechanism (not shown). Sharp edge 11 may be made of a biodegradable substance that can be detached from an endoscope 10, 60, such as by an accidental loss of tip 14 during use.

Preferably, penetrating endoscope 10 is specifically designed to penetrate tissues with its sharp leading edge 11. Leading edge 11 may be of various sharpness grades. To some degree, the penetrating ability is dependent on the amount of force the user applies to the device 10. Penetrating endoscope 10, by virtue of its sharper leading edge 11, decreases or eliminates trauma that a conventional endoscope having a blunt end may cause if forced into and through tissues. The sharper edge endoscope 10 reduces the slashing, crushing and other tissue deforming injuries that would result from attempting to use a conventional endoscope. In addition, such tissue injuries would impact diagnosis, and would create tissue damage, bleeding, and wound healing problems.

Tip 11, 14 of penetrating endoscope 10, and endoscope rod 6 are each made to withstand pressure and mechanical stress placed upon it. For example, after penetrating the tissues, penetrating endoscope 10 must have a structural integrity allowing subsequent use for visualizing and manipulating the body tissues such as during magnification, staining, deposit of materials and biopsy.

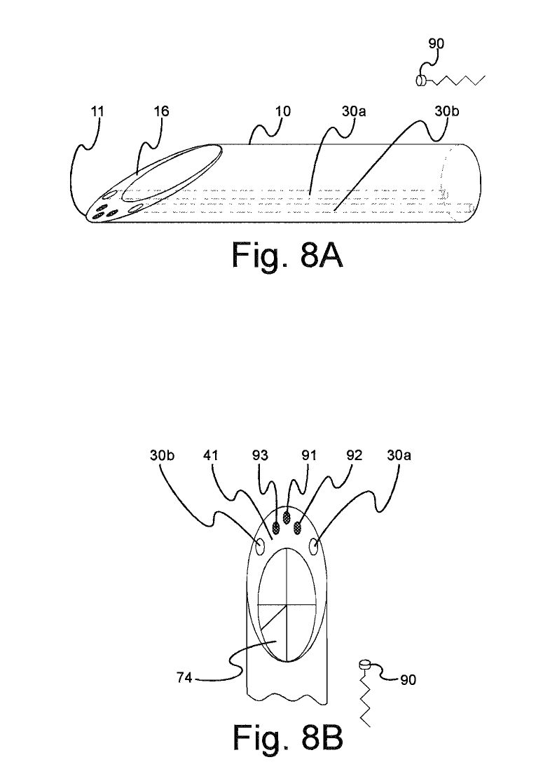

FIGS. 8A-8B schematically show another embodiment of a penetrating endoscope 10 adapted for use with an integrated staining system and/or cauterization system. A high performance is achieved by penetrating endoscope 10 having built-in channels 30a, 30b respectively for inflow and outflow, and having structure for visual diagnosis, biopsy, material deposit, and cautery. Although shown as a single flat surface, distal end surface 16 may be formed as a “hooded” compartment having hooded sides angled inwardly toward a longitudinal axis of endoscope 10, such angle preferably being contoured so that various components mounted along such a hooded type of surface 16 are thereby angled to perform at an optimum relative location. In such a case, a leading edge 11 may extend past a lens 74 and in so doing effect a chamber 41 having depth measured from lens 74 to a leading edge 11. For cautery, a monopolar electrode 91 and distant ground 90 are employed. Alternatively or additionally, bipolar electrodes 92, 93 may be employed.

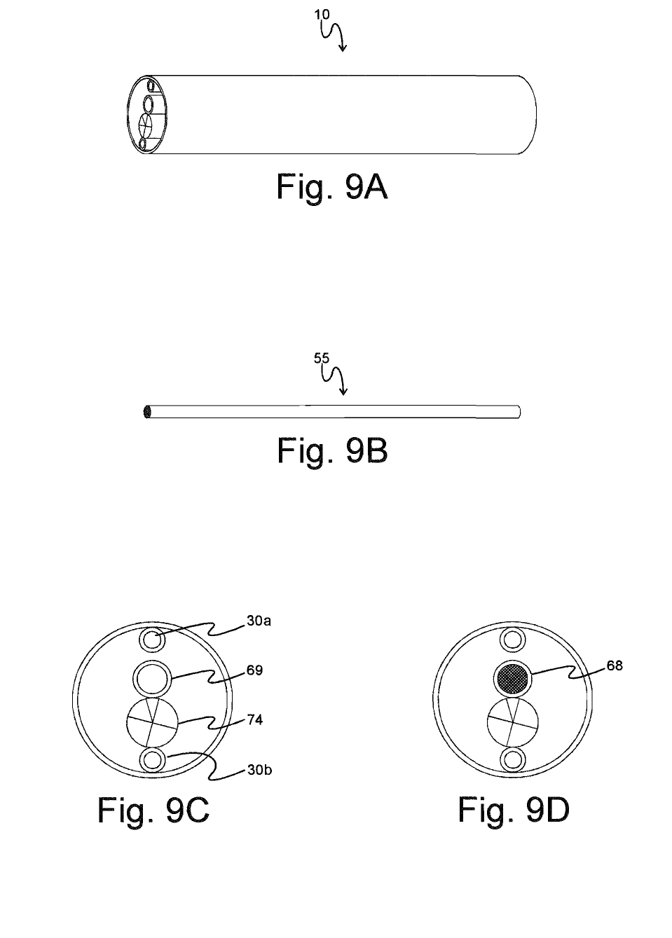

FIGS. 9A-9D, schematically show another embodiment of a penetrating endoscope 10 having a working channel 69, for example enclosing a removably insertable device 68 such as a cautery system rod or forceps for biopsy purposes. A corresponding endoscopic biopsy method improves over conventional macroscopic methods by allowing biopsy under microscopic visual control. Such is also an improvement over conventional contact endoscopy. A partitioned or segmented lens 74 may be structured and used in a manner similar to lenses 71, 72, previously described. As shown simply in FIG. 9B, a cautery rod 55 is typically formed with an elongated tubular shape.

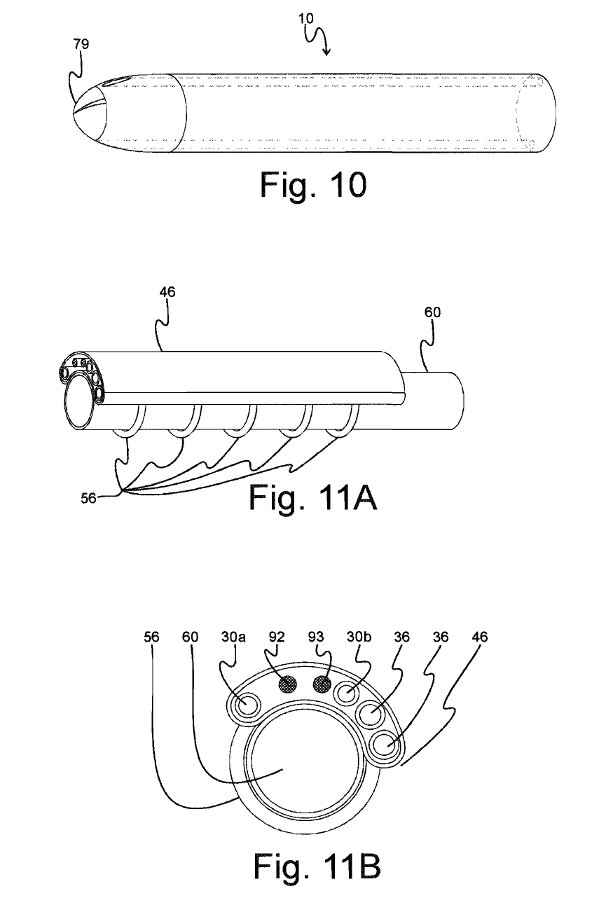

FIG. 10 schematically shows an embodiment of a penetrating endoscope 10 having a rounded lens tip 79 that is easily passed through tissues compared with a flat-tipped endoscope tip. Rounded lens 79 may have multiple focal and magnification sections 711-715, as in the previously described example of FIG. 2A. This endoscope 10 does not utilize a chamber in its construction, but can create a chamber effect by a method of first pushing into tissue and then withdrawing slightly. Upon withdrawing, a space is temporarily created in tissue and is then used as an ersatz chamber, such as for staining.

It is noted that the embodiment shown in FIG. 12 may be adapted for use in a distal end of a penetrating endoscope 10, where stepped tip 80 tip is streamlined in a series of lens steps 81-85. An overall tapering of endoscope tip 80 allows endoscope 10 to pass through tissues with reduced resistance. Steps 81-85 each act as a lens, and such may be lenses of the same or different focus and magnification, as previously described.

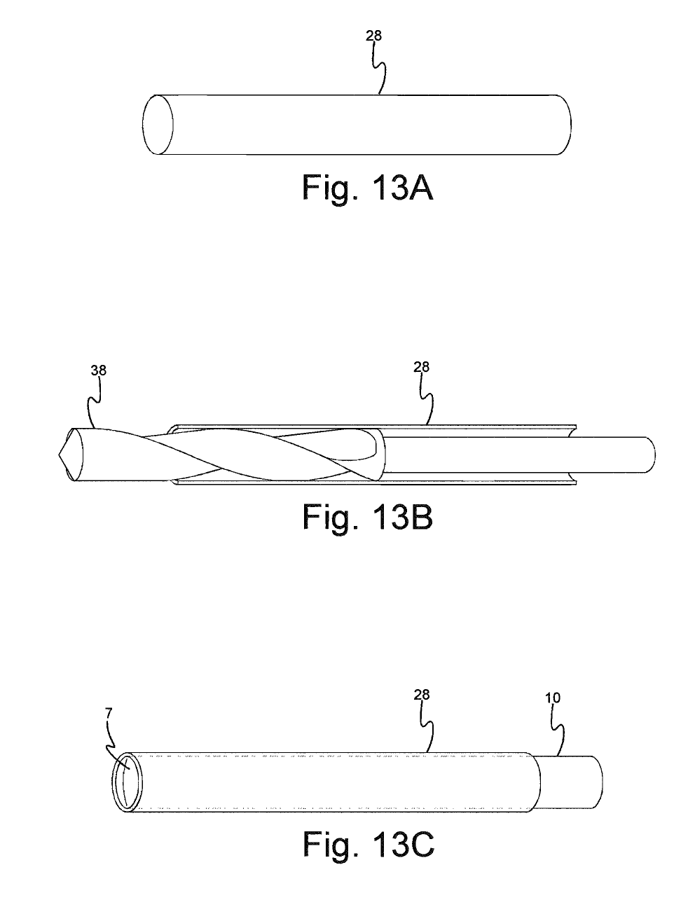

FIGS. 13A-13C schematically show a bone penetration type endoscope system that includes a sheath 28 having a structure for enclosing either a drill bit 38 passing therethrough, or enclosing an endoscope 60, 10. For example, a penetrating endoscope 10 may be adequate for penetrating soft tissues, but harder tissues such as bone and cartilage may necessitate that a drill be used. For pathological diagnosis to take place within the marrow of bone, inside the skull, or inside the paranasal sinuses, a bone barrier must first be breached. In one embodiment, penetrating endoscope 10 may be inserted into sheath 28 for penetrating tissue while viewing. Upon contact with a hard tissue surface, penetrating endoscope 10 is withdrawn from sheath 28 and drill bit 38 is placed through sheath 28. Next, drill bit 38 rotatably perforates the hard tissue and provides access to deeper tissues. Drill bit 38 is then withdrawn and endoscope 10 is again placed into sheath 28 and moved forward into the newly accessed tissues. Penetrating endoscope 10 is then used to complete the In-Vivo Pathology Diagnosis. Alternatively, a trocar may be used instead of a drill.

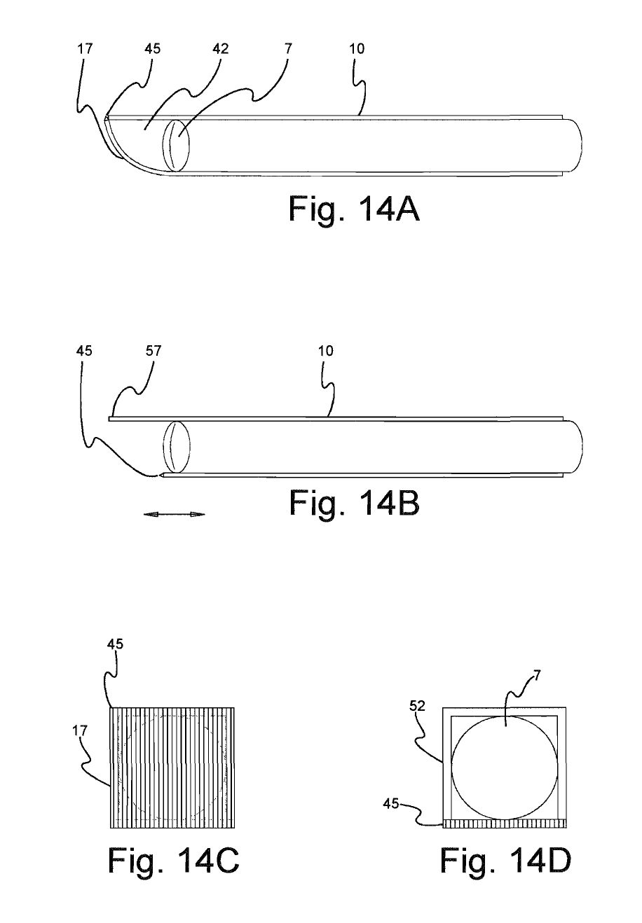

FIGS. 14A-14D schematically show a retractable tip 17 that may be adapted for use with penetrating endoscope 10 and/or penetrating sheath 20. Retractable tip 17 includes a retractable leading edge 45. In one example, during penetration of tissues, retractable tip 17 is placed in a closed position as shown in FIG. 14A, with leading edge 45 being closed to cover the lens portion 7 of endoscope 10. Leading edge 45 is preferably contoured and/or angled to create a streamlined tip. FIGS. 14C and 14D respectively schematically show front views of a retractable tip 17 in a closed position and in an open position. A retractable tip guide 57 may be formed in a suitable shape for allowing tip 17 to move between open and closed positions. A closed position acts to protect lens area 7 from becoming blurred or blocked, and reduces contamination of chamber 42. In one example, penetrating endoscope 10 has a closed tip 17 and is manipulated by the user to penetrate the tissues and arrive at the target area.

Thereafter, the user retracts moveable tip 17 structures as shown in FIG. 14B and exposes the desired site to staining, biopsy, endoscopic visualization, cautery, and/or others—the methods of In Vivo Pathology Diagnosis. In one optional additional procedure, while tip 17 is being re-closed, sharp leading edge 45 may be used to remove a biopsy tissue sample that is enclosed in chamber 42. In such a case, endoscope 10 is moved forward in an open position to be in contact with body tissues, whereupon the closing of tip 17 severs tissue. By closing, a piece of tissue is cut away from the tissue bed by the sharp edge 45 and is retained by a closed retractable tip 17 in chamber area 42. When penetrating endoscope 10 is subsequently withdrawn from the body, the tissue biopsy sample is removed from chamber 42 for future analysis.

FIG. 15 schematically shows an endoscope or sheath shape 87 that uses an overall cork-screw form adapted to enable its tissue penetration. For example, by rotating endoscope 10 about its longitudinal axes during insertion, corresponding tissues are parted and a relatively atraumatic penetration of the tissues results.

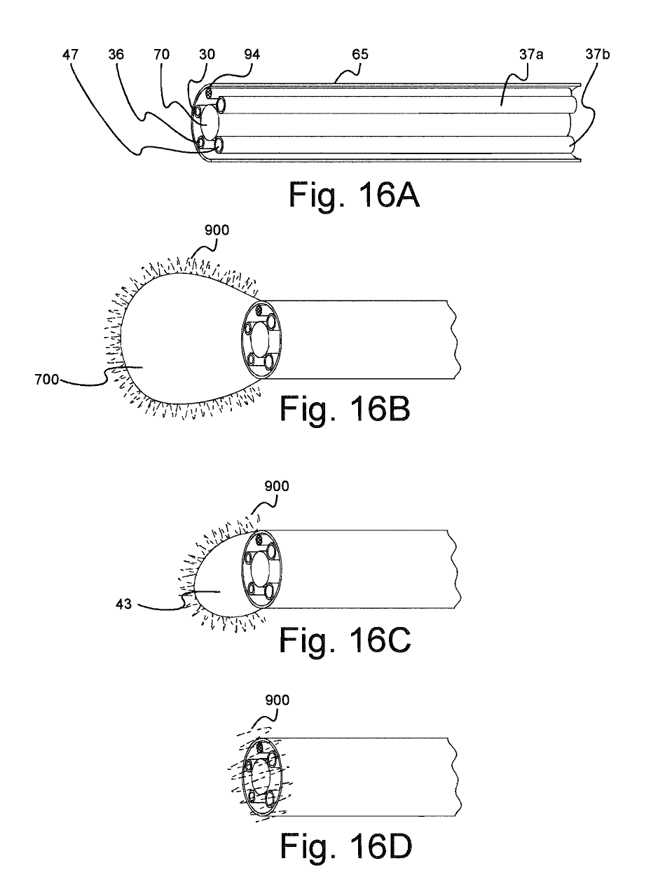

FIGS. 16A-16D schematically show a penetrating endoscope 64 having a structure adapted for insufflation of air, a specific gas, or a stain, liquid or gel through respective insufflation and deflation channels 37a, 37b of endoscope 64. Although described for an endoscope, such structure may alternatively be implemented in a penetrating sheath 20. Such sheath 20 may include a chamber 40, for example as shown in FIGS. 3A-3B. Endoscope 64 has a separate irrigation channel 30 and a channel 36 that may be used for additional irrigation process such as suction, or as a working channel. In the event such channel 30, 36 becomes blocked by tissue during the tissue penetration, it may be cleared by flushing with a liquid or by a mechanical guide wire passed through the blocked channel(s) 30, 36.

The aforementioned materials flow through channel 37a to the tip area 47. The insufflated air or other material 700 allows for better visualization of and dissection through tissues 900. By first insufflating, tissues are expanded away from endoscope tip 47, resulting in a space shown as air 700 in FIG. 16B. Upon deflation, such potential space 43 is left empty, and is subsequently used as an actual space, or chamber, for stain fluid irrigations and the like, as shown in FIG. 16C. The insufilated gas or other substance 700 may be placed in precise quantities and for precise durations. The control of the amount may be accomplished by eye-hand coordination, with the user deciding when the amount is sufficient. Alternately, an injection system (not shown) may supply precise dosages in conjunction with an external pump, controlled manually or by a computer program. Such pump, for example, may dispense gas or liquid material 700 in 0.5 cc increments. Channel 37b may remove material 700, such as by suction, either continuously or, for example, in a controlled manner synchronized with inflow of channel 37a. FIG. 16D shows tissue 900 that has just been stained and is available for visual diagnosis via lens 70. When IVPD procedures are completed, a cautery system 94 provided at tip area 47 may be used, as previously discussed.

Preferably, a particular penetrating sheath or penetrating/contact endoscope, and corresponding methods, are adapted for diagnostic use both in deep tissue and in superficial surface tissues of the body. For example, a sheath or endoscope may be pressed onto the skin for creating a chamber 40, and then channels, lens tips, stains and IVPD methods may be used for diagnosing a skin surface problem. Such tissue surface is either on the outside or inside of the body. Inside of the body, for example, a skin type surface being examined may be part of a colon, oral cavity, nasal tissue, esophagus, or stomach. Methods of In Vivo Pathology Diagnosis (IVPD) such as staining, viewing, and cautery are also applicable to these surface devices and procedures.

In addition, such IVPD methods and devices may be employed during an open surgical procedure, where target tissue is on the surface(s) of an exposed interior organ, for example an exposed liver. Such IVPD may include endoscopic placement of medications, chemicals and compounds, slow-release materials, nucleotides, viral and bacterial carriers, radioactive materials, micro-capsules and containers, clotting and anti-coagulation agents, and other substances.

The sheath and endoscope diameters will vary a great deal depending on application, i.e., the location to be accessed. Endoscopes and sheaths according to the present invention range from sub-millimeter diameters, such as for sialendoscopes, to supra-centimeter diameters, for example endoscopes adapted for gastro-intestinal IVPD.

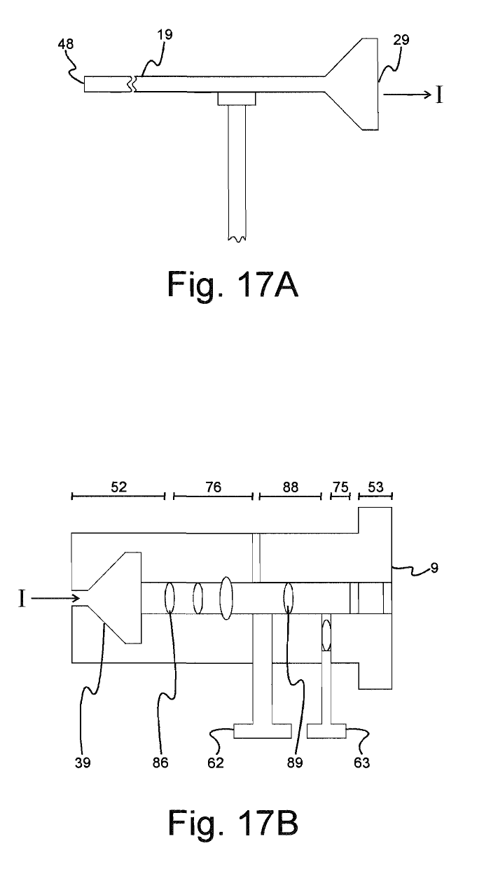

FIGS. 17A-17B schematically show an attachment 18 for a conventional endoscope 19 to transform it into a microscopic IVPD instrument. Components of attachment 18 include coupling members 52, 53 respectively disposed at opposite ends of attachment 18, for secure attachment onto the conventional endoscope 19 and onto video equipment such as cameras or other. For example, eyepiece end 29 of endoscope 19 is secured by a snug fitting 39 of attachment 18, the interconnection being shown by arrows “I”. The observer end 9 of attachment 18 can be used without a television camera being attached to coupler 53 by instead attaching an eyepiece (not shown) thereto. Such eyepiece allows the user to directly view all the way to the endoscope tip 48 when endoscope 19 is attached to attachment 18.

A magnification section 76 includes one or more fixed lenses 86 that preferably provide a lower level of magnification. Focus appliance 88 provides a clear view of the target by providing a manual knob 62 that is moved by the user for varying the optical focus. In addition, a variable magnification section 75 has one or more lenses 89 that may be inserted or removed from the optical path, thereby effecting a variable magnification. The observer may need to switch back and forth between different magnifications in order to correctly diagnose the pathology. Using a manual knob 63, the magnification can be increased or decreased through the movement within lens set 75. It is often desirable to obtain microscopic levels of magnification and, in such a case, magnification sections 75, 76 are adapted to provide magnification sufficient to accurately diagnose pathology of the target tissue without the need for performing a biopsy.

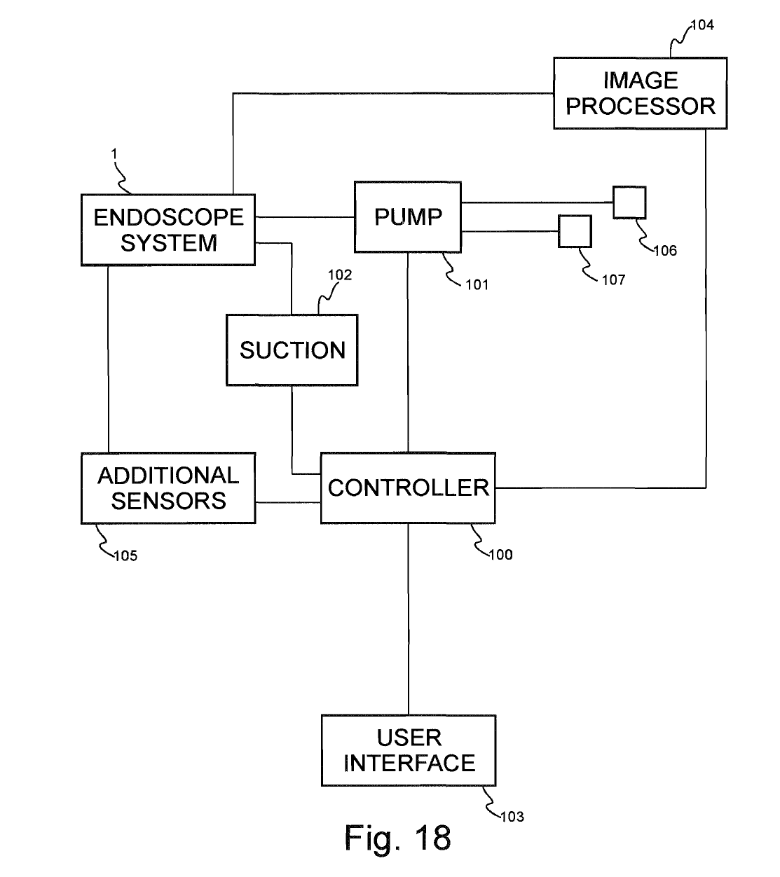

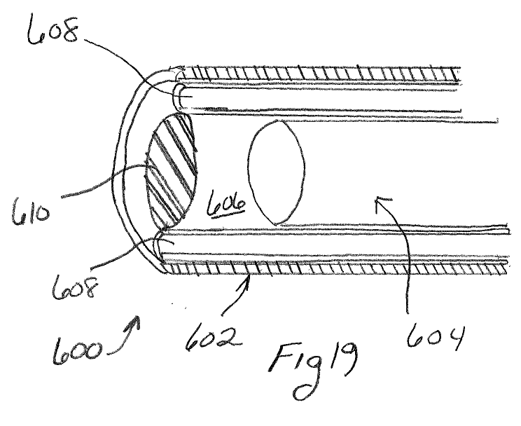

FIG. 18 schematically shows an exemplary computer controller 100 operative to control operations related to use of an endoscopy system 1. An endoscope system 1 has an irrigation channel 30a that receives a material 700 from a pump 101. As described herein above, a pumping action provides material 700 to a chamber 40, such as for a staining procedure. Material 700 may be provided from separate material sources 106, 107, for example bulk containers, pre-packaged dosages, and others.

A suction device 102, e.g., a vacuum pump, is sealingly attached to a suction channel 30b of system 1 for removal of fluids/materials from chamber 40. Pump 101 and suction device 102 are in communication with controller 100, for receiving control signals that change operation, and for outputting information to controller 100. An image processor 104, such as a processor of information from a video camera attached to an endoscope 10, 19, may optionally be in communication with controller 100. Additional sensors/instruments 105 may optionally also be in communication with controller 100.

A user interface 103 is attached to controller 100. In operation, controller 100 may include a computer program product residing on a computer readable medium having a plurality of instructions stored thereon which, when executed by controller 100, cause one or more processes to occur. In a manual mode, controller 100 controls operations of pump 101 and suction device 102 according to user inputs to interface 103, such as when a surgeon manually starts or stops such operations by pressing a control button (not shown) on interface 103. In an automated mode, controller 100 may cause, for example, controlled injection of individual measured pulses provided from source(s) 106, 107, controlled titration for variable dilution of a stain, etc. Such automated process may include manual inputs from a user, such as when starting or stopping a sequence of individual steps or by dynamically varying a concentration, timing of injection, time periods of injection, strength of suction, and others.

However, the extreme precision required in biological staining is compounded by greatly increased interactions and reactions of stains and associated materials with adjacent tissue during the in vivo staining process. As a result, additional sensors 105 are preferably used for monitoring pH, temperature, color, etc., and control of staining is preferably optimized by the computer program algorithms, for example flow rate of channels 30, temperature of materials 700, and sequencing of staining, counterstaining, and washing steps including exposure times. In fact, many processes require automated control. The automated steps are preferably adjusted by the user in small increments for tailoring the in vivo staining process according to results being viewed with associated real-time endoscopy, and by results of ancillary testing such as titration to determine dye content of used materials being suctioned from chamber 40. High volumes of blood, nutrients, and other materials 700 may also be injected to the target tissue or another injection site by peripheral equipment being controlled by controller 100, such as equipment in communication with controller 100 via serial communications links (not shown).

It is noted that, for simplification of this description, variations of individual apparatus and/or procedural steps described herein may have the same reference numbers even though they may embody different features and have similarities of varying degree.

Although the Applicant has disclosed the best mode of practicing the invention perceived presently by the Applicant, it is to be understood that specific disclosed embodiments are by way of example and are not limiting. Consequently, the reader will understand that variations and modifications exist within the scope and spirit of the present invention. It is intended that the appended claims be construed to include alternative embodiments to the extent permitted by the prior art.

What is claimed is:

1. An in vivo pathology diagnosis system, comprising:
a penetrating endoscope-type sheath having an inner surface, an inflow channel and an outflow channel, each channel having an opening at the inner surface proximate a distal end of the sheath, the sheath being structured for penetrating tissue when the sheath is pushed by an external force in a direction along the center longitudinal axis of the sheath;
a contact endoscope having a front lens and an outer surface;
a sealing member disposed between the inner surface of the sheath and the outer surface of the endoscope; and
a chamber formed as a variable space between the distal end of the sheath and the front lens of the contact endoscope,
wherein the contact endoscope is slidable along the inner surface of the sheath, thereby varying the space of the chamber.

2. The in vivo pathologic diagnosis system of claim 1, wherein the front lens has a plurality of lens segments having different magnification respecting one another.

3. The in vivo pathology diagnosis system of claim 1, wherein the front lens has a round side facing the chamber.

4. The in vivo pathology diagnosis system of claim 1, wherein the sealing member comprises an o-ring.

5. The in vivo pathology diagnosis system of claim 1, wherein the sheath has a pointed tip at its distal end, the pointed tip having an apex at a distance from the center longitudinal axis of the sheath.

6. The in vivo pathology diagnosis system of claim 5, wherein the distance is equal to a radius of the sheath.

7. The in vivo pathology diagnosis system of claim 5, wherein the front lens of the endoscope has a side facing the chamber, the side having a same shape as a shape of the pointed tip of the sheath.

8. The in vivo pathology diagnosis system of claim 1, wherein the sheath has a pointed tip at its distal end, the pointed tip having an apex along the center longitudinal axis of the sheath and having at least one portal opening between the apex and a lengthwise side of the sheath.

9. The in vivo pathology diagnosis system of claim 8, wherein the front lens of the contact endoscope has at least one lens side facing the corresponding at least one portal of the sheath.

10. The in vivo pathology diagnosis system of claim 1, further comprising a cauterizing system having, proximate the distal end of the sheath, at least one of a monopolar electrode and a bipolar electrode set, and having a ground electrode.

11. A method of in vivo pathology diagnosis, comprising:
providing a penetrating device that compresses at least one of a penetrating sheath and a penetrating endoscope, the penetrating device having an irrigation channel with an opening at a distal end chamber of the device;
penetrating tissue with the penetrating device until the distal end is proximate a target tissue area;
supplying stain to the chamber via the irrigation channel, thereby staining the target tissue area; and
diagnosing pathology of the stained target tissue by viewing such stained tissue through the penetrating device.

12. The method of claim 11, wherein the penetrating device has a stain removal channel, the method further comprising suctioning stain from the chamber via the stain removal channel.

13. The method of claim 12, wherein the supplying of stain to and suctioning of stain from the chamber are performed as a series of alternating events each having a corresponding flow time controlled by a computer program.

14. The method of claim 11, wherein the supplying of stain is performed as a sequence of pump events each causing movement of the stain toward the chamber.

15. The method of claim 11, further comprising advancing a contact endoscope in a direction toward the distal end of the penetrating device until a distal end of the contact endoscope touches the target tissue.

16. The method of claim 15, wherein the viewing of the stained target tissue is performed through a lens assembly located at the distal end of the contact endoscope.

17. The method of claim 16, wherein the lens assembly is formed as a plurality of segments each having a different magnification respecting one another, the method further comprising selecting one of the segments for the viewing of the stained tissue.

18. The method of claim 11, further comprising creating feedback control information by determining a degree of staining of the target tissue, and controlling the supplying of stain based on the feedback control information.

19. The method of claim 18, wherein the creating of feedback control information includes a user determination based on the viewing of the stained tissue.

20. The method of claim 11, wherein the penetrating device includes a washing channel, the method further comprising supplying wash solution to the chamber via the washing channel, thereby washing the chamber.

21. The method of claim 11, wherein the penetrating device is a contact endoscope.

22. The method of claim 11, wherein the staining and the viewing are performed simultaneously.

23. A method of in vivo pathology diagnosis comprising:
providing penetrating endoscope means having a sharp slanted tip at a distal end, the penetrating endoscope means being for forcibly penetrating tissue until the distal end is at a target tissue location; and
placing and depositing a material at the target tissue location via the penetrating endoscope.

24. The method of claim 23 wherein the material comprises at least one of a stain, radiotherapy pellet, a medication, a nucleotide and a microbe.

25. The method of claim 23, wherein the material comprises a stain, further comprising determining that the target tissue has been stained and then viewing the stained tissue via the penetrating endoscope means disposed at the target tissue location.

26. An in vivo pathology diagnosis system, comprising:
a penetrating endoscope sheath having an interior surface with an essentially cylindrical shape and a longitudinal axis, the penetrating endoscope sheath comprising:
a front tip member adapted for penetrating living tissue without substantial distortion of the cylindrical shape;
an irrigation channel formed essentially in parallel with the longitudinal axis and having an outlet port at the interior surface at a location adjacent the front tip member;
a suction channel formed essentially in parallel with the longitudinal axis and having an inlet port at the interior surface at a location adjacent the front tip member and opposing the irrigation channel outlet port;
at least one of a monopolar cauterizing electrode and a pair of bipolar cauterizing electrodes disposed adjacent the front tip member;
a contact endoscope having a front lens formed as a plurality of lens segments each having a different magnification, the front lens being adapted for contacting target tissue, the contact endoscope being disposed at least partially in the interior space and slidable along the longitudinal axis of the sheath;
a chamber formed as a variable space between the front tip member of the sheath and the front lens of the contact endoscope, where the contact endoscope is slidable along the inner surface of the sheath, thereby varying the space of the chamber, and where the inlet and outlet ports are in communication with the chamber when the contact endoscope is in a retracted position;
a stain for being input to the chamber via the irrigation channel to stain the target tissue; and
a controller for regulating flow of stain into and amount of suction out of the chamber,
wherein the endoscope in an extended position extends beyond the front tip member of the penetrating endoscope sheath, and wherein the stained target tissue is viewable via a selected one of the lens segments

27. An in-vivo pathology diagnosis system comprising
a penetrating endoscope sheath having an inner surface, an inflow channel and an outflow channel, each channel having an opening at the inner surface proximate to the distal end of the sheath, and an endoscope-receiving channel
a conventional endoscope having a distal end and an outer surface, the contact endoscope being axially moveable within the endoscope sheath receiving channel, and
an optical lens coupled to the distal end of the sheath, the optical lens being disposed in optical alignment with the distal end of the conventional endoscope when the endoscope is positioned proximate to the optical lens.

28. The in-vivo pathology diagnosis system of claim 27 wherein the optical lens comprises a corrective optical lens disposed radially inwardly of the inflow and outflow channels, and working channels.
